(19) United States
(12) Patent Application Publication
(10) Pub. No.: US 2019/0192437 A1
(43) Pub. Date: Jun. 27, 2019

(54) POWDER FORM AND USE OF HYDROGEL COMPOSITIONS FOR ALLEVIATING DEGENERATIVE JOINT AND TENDON TEAR

(71) Applicant: Industrial Technology Research Institute, Hsinchu (TW)

(72) Inventors: Hsin-Hsin SHEN, Zhudong Township (TW); Wen-Shiang CHEN, Taipei City (TW); Chueh-Hung WU, Taipei City (TW); Ming-Kuan SUN, Taipei City (TW); Yu-Chi WANG, New Taipei City (TW); Sen-Lu CHEN, Zhunan Township (TW); Wei-Lin YU, Zhubei City (TW); Lih-Tao HSU, Lujhu Township (TW); Shih-Ping LIN, Kaohsiung City (TW)

(73) Assignee: Industrial Technology Research Institute, Hsinchu (TW)

(21) Appl. No.: 15/855,260

(22) Filed: Dec. 27, 2017

(51) Int. Cl. A61K 9/14; A61K 9/06; A61K 35/16; A61K 35/19; A61K 31/704; A61K 38/18; A61K 38/38; A61K 41/00; A61M 37/00

(52) U.S. Cl. CPC A61K 9/146; A61K 9/145; A61K 9/06; A61K 35/16; A61K 35/19; A61M 2037/0007; A61K 38/1841; A61K 38/385; A61K 41/0047; A61M 37/0092; A61K 31/704

(57) ABSTRACT

The disclosure provides a powder form and a hydrogel composition for alleviating degenerative joint and tendon tear. The powder form consists essentially of a 120-380 parts by weight biodegradable copolymer; 15-75 parts by weight urea; and 100 parts by weight platelet-rich plasma; wherein the biodegradable copolymer has a structure of formula (I) or formula (II):

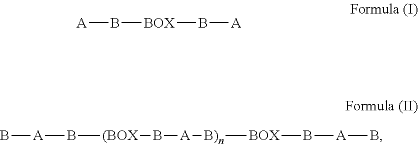

wherein A comprises a hydrophilic polyethylene glycol polymer; B comprises a hydrophobic polyester polymer; BOX is bifunctional group monomer of 2, 2′-bis(2-oxazoline) used for coupling the blocks AB or BAB; and n is an integer greater than or equal to 0.

### TECHNICAL FIELD

The technical field relates to a powder form, and in particular, it relates to a powder form having a therapeutic agent for the use of slowing joint degeneration and repairing tendon rupture.

### BACKGROUND

Degeneration is an unavoidable process that everyone will experience during their lifetime. Degeneration is often accompanied by pain and inflammation, thereby reducing the quality of life of patients. Common degenerative diseases caused by aging or excessive use are including degenerative arthritis and tendonopathy. Degenerative arthritis of the knee joint can cause cartilage wear, inflammation, and deformation of the joints, thereby causing significant pain and inconvenience to the patient.

Methods used clinically for treating degenerative diseases include hyaluronic acid injection, which injects a mucous-like macromolecular-hyaluronic-acid-containing agent, serving as a lubricant, into the knee joint to prevent joints or tendons from wearing, to inhibit inflammation, to stimulate secretion of synovial fluid, and to reduce joint pain. The declared effects, however, have not yet been supported by strong scientific evidence. It should be noted that the injected hyaluronic-acid can be spread over the non-affected region due to the fluidity of hyaluronic acid, resulting in a reduced therapeutic efficacy.

Therefore, there is a need to develop a novel slow-releasing therapeutic agent, which can be attached to defects or lesions of the joints or tendons, for use in treatment of joints and tendons.

### SUMMARY

According to embodiments of the disclosure, the disclosure provides a powder form, wherein the powder form consists essentially of 120-380 parts by weight of biodegradable copolymer, 15-75 parts by weight of urea, and 100 parts by weight of platelet-rich plasma (PRP). The biodegradable copolymer has a structure of Formula (I) or Formula (II):

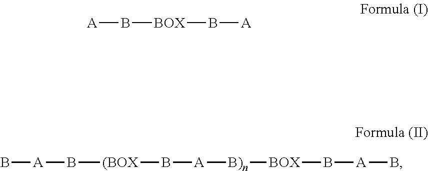

wherein A is a hydrophilic polyethylene glycol polymer; B is a hydrophobic polyester polymer; BOX is a bifunctional group monomer of 2, 2′-bis(2-oxazoline) used for coupling the blocks A-B or B-A-B; and n is 0 or an integer greater than 0.

According to embodiments of the disclosure, the disclosure further provides a use of hydrogel composition for alleviating degenerative joint and tendon tear, wherein the hydrogel composition includes 100 parts by weight of therapeutic agent and 120-380 parts by weight of biodegradable copolymer. The therapeutic agent includes platelet-rich plasma (PRP), doxorubicin, transforming growth factor, or a combination thereof, wherein the biodegradable copolymer has a structure of Formula (I) or Formula (II):

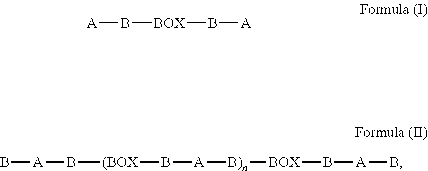

wherein A is a hydrophilic polyethylene glycol polymer; B is a hydrophobic polyester polymer; BOX is a bifunctional group monomer of 2, 2′-bis(2-oxazoline) used for coupling the blocks A-B or B-A-B; and n is 0 or an integer greater than 0.

A detailed description is given in the following embodiments with reference to the accompanying drawings.

### BRIEF DESCRIPTION OF THE DRAWINGS

FIG. 1 is a schematic view of cell co-culture system according to an embodiment of the disclosure;

FIGS. 2A and 2B are graphs plotting the cumulative release of transforming growth factor (TGF-b1) in the platelet-rich plasma (PRP) system or the platelet-rich plasma (PRP)/mPEG-PLGA biblock polymer system against time;

FIG. 3 is a graph plotting the human dermal fibroblasts cell (HDF) proliferation in various transforming growth factor (TGF-b1) sources;

FIGS. 4A and 4B are graphs plotting the human dermal fibroblasts cell (HDF) proliferation in the platelet-rich plasma (PRP) system, or the platelet-rich plasma (PRP)/mPEG-PLGA biblock polymer system against time;

FIG. 5 is a graph showing the TGF-b1 relative concentration at room temperature after mixing the platelet-rich plasma (PRP) with mPEG-PLGA biblock polymer;

FIG. 6A is a graph showing the TGF-b1 relative concentration of platelet-rich plasma (PRP) in various preservation conditions;

FIG. 6B is a graph showing the human dermal fibroblasts cell (HDF) proliferation of platelet-rich plasma (PRP) in various preservation conditions;

FIG. 7 is a graph showing the cumulative release percentage of transforming growth factor (TGF-b1) in platelet-rich plasma (PRP)/mPEG-PLGA biblock polymer system with various concentration against time;

FIG. 8 is a graph showing the cumulative release amount of doxorubicin in mPEG-PLGA biblock polymer system against time;

FIG. 9 is a graph showing the cumulative release amount of transforming growth factor (TGF-b1) in mPEG-PLGA biblock polymer system against time;

FIG. 10 is a schematic view of an in vitro ultrasound stimulation device according to an embodiment of the disclosure;

FIG. 11 is a graph plotting the doxorubicin release amount (by treating with the in vitro ultrasound stimulation device) against time;

FIG. 12 is a graph plotting the transforming growth factor (TGF-b1) release amount (by treating with the in vitro ultrasound stimulation device) against time;

FIG. 13 is a graph plotting the bovine serum albumin release amount (by treating with the in vitro ultrasound stimulation device) against time; and

FIGS. 14A-14C show the stagnation results in the drug-containing hydrogel, hyaluronic acid and physiological saline analyzed by visual appearance, IVIS spectrum and ultrasound imaging.

### DETAILED DESCRIPTION

The disclosure provides a powder form, wherein the powder form consists essentially of 120-380 parts by weight of biodegradable copolymer, 15-75 parts by weight of urea, and 100 parts by weight of platelet-rich plasma (PRP). The biodegradable copolymer has a structure of Formula (I) or Formula (II):

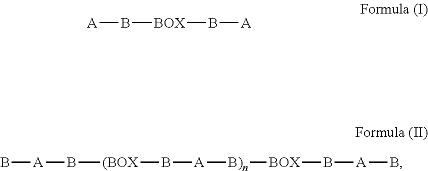

wherein A is a hydrophilic polyethylene glycol polymer; B is a hydrophobic polyester polymer; BOX is a bifunctional group monomer of 2, 2′-bis(2-oxazoline) used for coupling the blocks A-B or B-A-B; and n is 0 or an integer greater than 0.

According to embodiments of the disclosure, A can be polyethylene glycol (PEG), or methoxy-poly(ethylene glycol (mPEG).

According to embodiments of the disclosure, B can be poly(lactide-co-glycolide) (PLGA), poly(propionic-co-lactic (PPLA), poly(valeric-co-lactic) (PVLA), or poly(caproic-co-lactic (PCLA). The hydrophobic polyester polymer can have a molecular weight of 500-5000 g/mole. The term molecular weight disclosed in the disclosure means weight average molecular weight.

The biodegradable copolymer can be PEG-PLGA, PEG-PLGA-PEG, PLGA-PEG-PLGA, or combinations thereof. The term PEG means a hydrophilic polyethylene glycol (PEG) polymer, such as polyethylene glycol (PEG), or methoxy-poly(ethylene glycol) (mPEG)(methoxy-poly(ethylene glycol), mPEG). The hydrophilic polyethylene glycol (PEG) polymer can have a molecular weight of between 350-2000 g/mole. The term PLGA means hydrophobic poly(lactic-co-glycolic acid), and can be derived from D,L-Lactide, D-Lactide, L-Lactide, D,L-Lactic acid, D-Lactic acid, L-Lactic acid, glycolide, β-propiolactone, δ-valerolactone, or ε-caprolactone, such as poly(lactide-co-glycolide) (PLGA), poly(propionic-co-lactic (PPLA), poly(valeric-co-lactic) (PVLA), or poly(caproic-co-lactic) (PCLA). The hydrophobic poly(lactic-co-glycolic acid) can have a molecular weight of between 1000-3500 g/mole.

According to embodiments of the disclosure, the disclosure provides a use of hydrogel composition for alleviating degenerative joint and tendon tear, wherein the hydrogel composition includes 100 parts by weight of therapeutic agent and 120-380 parts by weight of biodegradable copolymer, wherein the therapeutic agent includes platelet-rich plasma (PRP), doxorubicin, transforming growth factor (TGF-b1), bovine serum albumin, or a combination thereof. The biodegradable copolymer has a structure of Formula (I) or Formula (II):

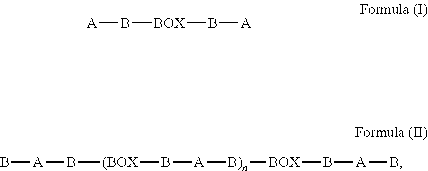

wherein A is a hydrophilic polyethylene glycol polymer; B is a hydrophobic polyester polymer; BOX is a bifunctional group monomer of 2, 2′-bis(2-oxazoline) used for coupling the blocks A-B or B-A-B; and n is 0 or an integer greater than 0.

According to embodiments of the disclosure, A can be polyethylene glycol (PEG), or methoxy-poly(ethylene glycol (mPEG).

According to embodiments of the disclosure, B can be poly(lactide-co-glycolide) (PLGA), poly(propionic-co-lactic (PPLA), poly(valeric-co-lactic) (PVLA), or poly(caproic-co-lactic (PCLA).

According to embodiments of the disclosure, the therapeutic agent of the disclosure includes platelet-rich plasma (PRP) and doxorubicin, wherein the weight ratio of the platelet-rich plasma (PRP) to the doxorubicin is 1:2.

According to embodiments of the disclosure, the therapeutic agent of the disclosure includes platelet-rich plasma (PRP) and transforming growth factor, wherein the weight ratio of the platelet-rich plasma (PRP) to the transforming growth factor is 1:2.

According to embodiments of the disclosure, the therapeutic agent of the disclosure includes platelet-rich plasma (PRP) and bovine serum albumin, wherein the weight ratio of the platelet-rich plasma (PRP) to the bovine serum albumin is 1:2.

According to embodiments of the disclosure, the use of hydrogel composition for alleviating degenerative joint and tendon tear of the disclosure further includes an ultrasonic treatment, which forces the hydrogel composition to release the therapeutic agent. In addition, the output intensity of the ultrasonic treatment can be 100-10000 W/cm². The ultrasonic treatment has a treatment period from 1 min to 5 min. Under the ultrasound stimulation, the release amount of the therapeutic agent can be increased.

According to embodiments of the disclosure, the use of hydrogel composition for alleviating degenerative joint and tendon tear of the disclosure, the hydrogel composition further includes 50-400 parts by weight of water.

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity.

### Preparation Example 1

First, 10.04 g of mPEG (methoxy poly(ethylene glycol)(with a molecular weight of 550 g/mole), 20 g of lactide, and 5.64 g of glycolide were subsequently added into a reactor, and the reaction bottle was heated slowly to force that the components were completely dissolved in the solvent. After heating to 160° C., the stannous 2-ethyl-hexanoate (14.0 μl) as catalyst was added into the reaction bottle. After heating for 8 hr, poly(lactide-co-glycolide) (PLGA) was obtained via polymerization of lactide and glycolide. After the reaction was completed, 1.84 g of succinic anhydride (SA) (with a molecular weight of 100.07 g/mole) was added into the reaction bottle. Next, after reacting for 4 hr, 1.28 g of 2, 2′-bis(2-oxazoline) (BOX)(with a molecular weight of 140.14 g/mole) was added into the reaction bottle. After the components were completely dissolved in the solvent, stannous octoate as catalyst was added into the reaction bottle. After reacting for 4 hr, the result (translucent gel) was reprecipitated with a solution (including diethyl ether and n-hexane, and the volume ratio of diethyl ether to n-hexane is 1:9). The result was washed three times to remove the residual monomers and dried in a vacuum for 24 hr at a temperature of 40° C., thus obtaining mPEG-PLGA biblock polymer.

### Preparation Example 2

Preparation of Platelet-Rich Plasma (PRP)

100 mL of pig blood was treated with a platelet rich plasma extraction kit (manufactured by Biosafe) and a cell separation system (manufactured by Sepax) (with a centrifugation time of 15 min and a centrifugation speed of 3400 rpm), obtaining a platelet-rich plasma (PRP).

### Preparation Example 3

A solution including 0.5 mL of mPEG-PLGA biblock polymer of Preparation Example 1 and urea was cooled to −20° C. to form a powder form, wherein the urea concentration of the powder form was 2%.

### Preparation Example 4

100% PRP Sample Solution (without the mPEG-PLGA Biblock Polymer)

The platelet-rich plasma prepared by Preparation Example 1 served as 100% PRP sample solution.

### Preparation Example 5

100% PRP Sample Solution (Including the mPEG-PLGA Biblock Polymer)

The powder form of Preparation Example was dispersed in a microcentrifuge tube, and 0.5 mL of 100% PRP sample solution of Preparation Example 3 was added into the tube to mix with the powder from via a tube oscillator. After standing for 20 min and then resolving, 100% PRP sample solution (including the mPEG-PLGA biblock polymer) was obtained.

### Preparation Example 6

50% PRP Sample Solution (without the mPEG-PLGA Biblock Polymer)

1 mL of 100% PRP sample solution (without the mPEG-PLGA biblock polymer) of preparation Example 4 was diluted with 1 mL of phosphate buffered saline (PBS), obtaining 50% PRP sample solution (without the mPEG-PLGA biblock polymer).

### Preparation Example 7

50% PRP Sample Solution (Including the mPEG-PLGA Biblock Polymer)

100 mg of powder form of Preparation Example 3 was dispersed in a microcentrifuige tube, and 0.5 mL of 50% PRP sample solution of Preparation Example 5 was added into the tube to mix with the powder from via a tube oscillator. After standing for 20 min and then resolving, 50% PRP sample solution (including the mPEG-PLGA biblock polymer) was obtained.

### Preparation Example 8

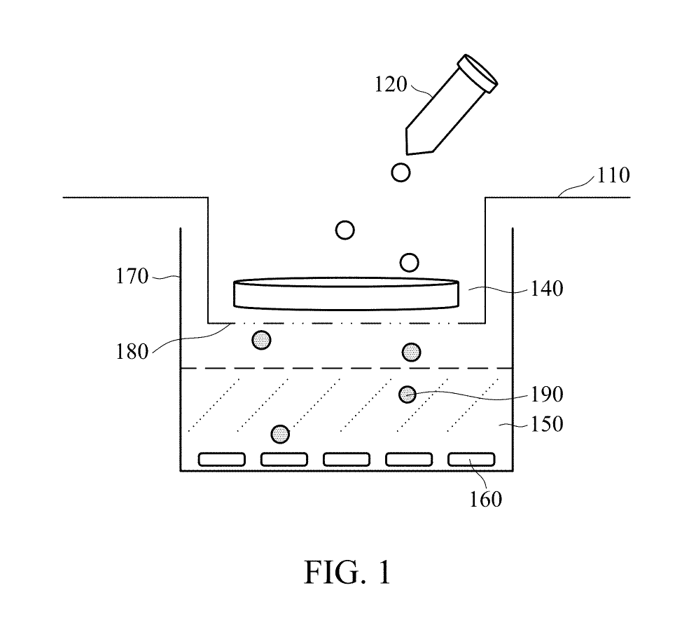

FIG. 1 is a schematic view of cell co-culture system. The experimental results were obtained by analyzing the culture fluid of the cell co-culture, in some examples of the disclosure.

First, 0.1 mL of 100% PRP sample solution 120 (without the mPEG-PLGA biblock polymer) of Preparation Example 4 was added into a multiple well plate 110. After the sample solution 140 was transferred into a gel, human dermal fibroblasts cells (HDF) 160 were implanted in the cell culture plate 170. 0.6 ml of medium (DMEM) 150 was added. Next, the multiple well plate 110 was hung on the cell culture plate 170, and the co-culture was performed at 37° C. (as shown in FIG. 1). The transforming growth factor (TGF-b1) 190 released from PRP was slowly penetrated into the cell culture plate 170 via the hole 180 (with a diameter of 0.4 μm) in the bottom of the multiple well plate 110. After the reaction was completed, 0.6 mL of culture fluid 150 was sampled and analyzed.

### Preparation Example 9

First, 0.1 mL of 100% PRP sample solution (including the mPEG-PLGA biblock polymer) 120 of Preparation Example 5 was added into a multiple well plate 110. After the sample solution 140 was transferred into a gel, human dermal fibroblasts cells (HDF) 160 were implanted in the cell culture plate 170. 0.6 ml of medium (DMEM) 150 was added. Next, the multiple well plate 110 was hung on the cell culture plate 170, and the co-culture was performed at 37° C. (as shown in FIG. 1). The transforming growth factor (TGF-b1) 190 released from PRP was slowly penetrated into the cell culture plate 170 via the hole 180 (with a diameter of 0.4 μm) in the bottom of the multiple well plate 110. After the reaction was completed, 0.6 mL of culture fluid 150 was sampled and analyzed.

### Preparation Example 10

First, 0.1 mL of 50% PRP sample solution 120 (without the mPEG-PLGA biblock polymer) of Preparation Example 6 was added into a multiple well plate 110. After the sample solution 140 was transferred into a gel, human dermal fibroblasts cells (HDF) 160 were implanted in the cell culture plate 170. 0.6 ml of medium (DMEM) 150 was added. Next, the multiple well plate 110 was hung on the cell culture plate 170, and the co-culture was performed at 37° C. (as shown in FIG. 1). The transforming growth factor (TGF-b1) 190 released from PRP was slowly penetrated into the cell culture plate 170 via the hole 180 (with a diameter of 0.4 μm) in the bottom of the multiple well plate 110. After the reaction was completed, 0.6 mL of culture fluid 150 was sampled and analyzed.

### Preparation Example 11

First, 0.1 mL of 50% PRP sample solution 120 (including the mPEG-PLGA biblock polymer) of Preparation Example 7 was added into a multiple well plate 110. After the sample solution 140 was transferred into a gel, human dermal fibroblasts cells (HDF) 160 were implanted in the cell culture plate 170. 0.6 ml of medium (DMEM) 150 was added. Next, the multiple well plate 110 was hung on the cell culture plate 170, and the co-culture was performed at 37° C. (as shown in FIG. 1). The transforming growth factor (TGF-b1) 190 released from PRP was slowly penetrated into the cell culture plate 170 via the hole 180 (with a diameter of 0.4 μm) in the bottom of the multiple well plate 110. After the reaction was completed, 0.6 mL of culture fluid 150 was sampled and analyzed.

### Preparation Example 12

First, 0.1 mL of 100% PRP sample solution (without the mPEG-PLGA biblock polymer) of Preparation Example 4 was added into a multiple well plate. After the sample solution was transferred into a gel, 0.6 mL of 0.6 mL of medium (DMEM) was implanted in the cell culture plate. Next, the multiple well plate 110 was hung on the cell culture plate 170. The transforming growth factor (TGF-b1) released from PRP was slowly penetrated into the cell culture plate via the hole (with a diameter of 0.4 μm) in the bottom of the multiple well plate. After the reaction was completed, 0.6 mL of culture fluid was sampled and analyzed.

### Preparation Example 13

First, 0.1 mL of 100% PRP sample solution (including the mPEG-PLGA biblock polymer) of Preparation Example 5 was added into a multiple well plate. After the sample solution was transferred into a gel, 0.6 mL of medium (DMEM) was implanted in the cell culture plate. Next, the multiple well plate 110 was hung on the cell culture plate 170. The transforming growth factor (TGF-b1) released from PRP was slowly penetrated into the cell culture plate via the hole (with a diameter of 0.4 m) in the bottom of the multiple well plate. After the reaction was completed, 0.6 mL of culture fluid was sampled and analyzed.

### Preparation Example 14

First. 0.1 mL of 50% PRP sample solution (without the mPEG-PLGA biblock polymer) of Preparation Example 6 was added into a multiple well plate. After the sample solution was transferred into a gel, 0.6 mL of medium (DMEM) was implanted in the cell culture plate. Next, the multiple well plate 110 was hung on the cell culture plate 170. The transforming growth factor (TGF-b1) released from PRP was slowly penetrated into the cell culture plate via the hole (with a diameter of 0.4 μm) in the bottom of the multiple well plate. After the reaction was completed, 0.6 mL of culture fluid was sampled and analyzed.

### Preparation Example 15

First, 0.1 mL of 50% PRP sample solution (including the mPEG-PLGA biblock polymer) of Preparation Example 7 was added into a multiple well plate. After the sample solution was transferred into a gel, 0.6 mL of medium (DMEM) was implanted in the cell culture plate. Next, the multiple well plate 110 was hung on the cell culture plate 170. The transforming growth factor (TGF-b1) released from PRP was slowly penetrated into the cell culture plate via the hole (with a diameter of 0.4 μm) in the bottom of the multiple well plate. After the reaction was completed, 0.6 mL of culture fluid was sampled and analyzed.

### Example 1

Transforming Growth Factor (TGF-b1) Release Test

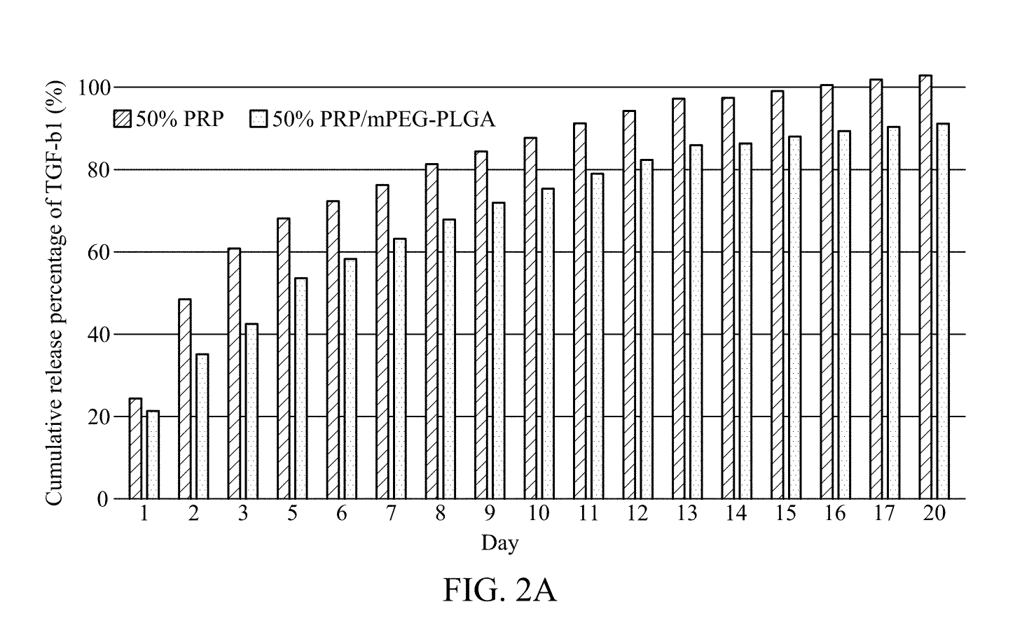

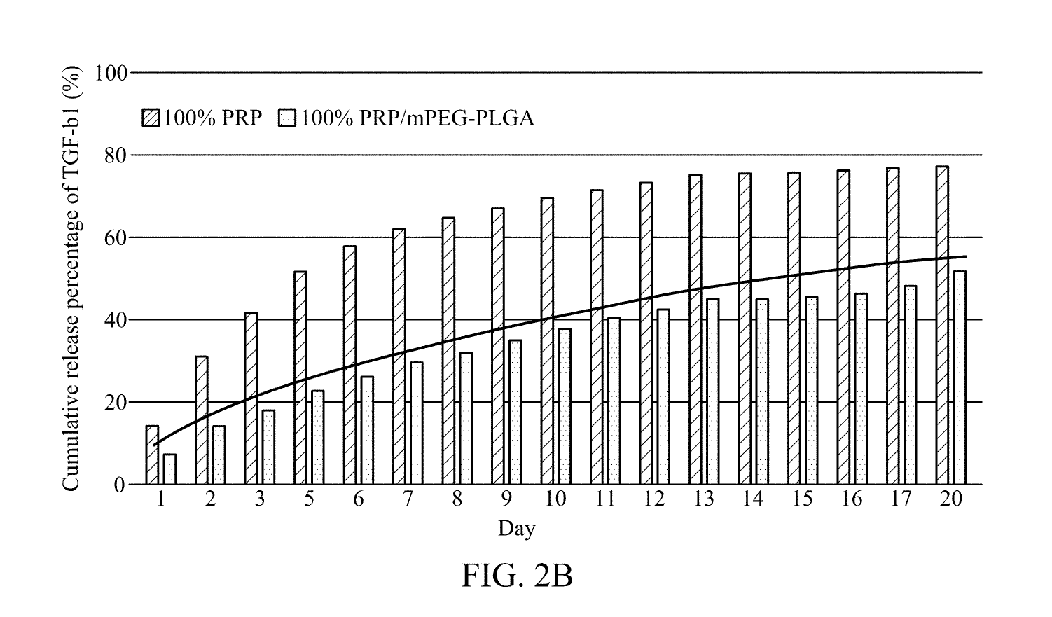

0.6 mL of culture fluid was extracted from the cell culture plates of Preparation Examples 12-15 individually after reacting 24 hr, and then stored at −20° C. Next, 0.6 mL of medium (DMEM) was implanted in the cell culture plate of Preparation Examples 12-15, and then 0.6 mL of culture fluid was extracted from the cell culture plates of Preparation Examples 12-15 individually after reacting 24 hr. and then stored at −20° C. The aforementioned steps were repeated until 20 days. Finally, the release amount of the TGF-b1 of the culture fluids were determined by enzyme-linked immunosorbent assay (ELISA), and the results are shown in FIG. 2A and FIG. 2B.

As shown in FIGS. 2A and 2B, when PRP is covered by mPEG-PLGA biblock polymer (mPEG-PLGA), the release amount of transforming growth factor (TGF-b1) released from PRP would not be affected by mPEG-PLGA and exhibits slow release effect. Taking 50% PRP sample solution as example, TGF-b1 of 50% PRP sample solution without the mPEG-PLGA biblock polymer would be completely released out after 17-20 days. Furthermore, after 20 days, there are 10% 50% TGF-b1 in PRP sample solution including the mPEG-PLGA biblock polymer (50% PRP/mPEG-PLGA). It proves that mPEG-PLGA biblock polymer can enhance the slow-release ability of PRP for releasing transforming growth factor. The slow release effect is more obvious in the group including 100% PRP and 100% PRP/mPEG-PLGA. When the total release amount of TGF-b1 is regarded as 100%, the cumulative release amount of TGF-b1 in 100% PRP sample solution (without the mPEG-PLGA biblock polymer) is about 14% at the first day, and the cumulative release amount of TGF-b1 in 100% PRP sample solution (including the mPEG-PLGA biblock polymer) is about 7% at the first day. Furthermore, the cumulative release amount of TGF-b1 in 100% PRP sample solution (without the mPEG-PLGA biblock polymer) is about 68% at the tenth day, and the cumulative release amount of TGF-b1 in 100% PRP sample solution (including the mPEG-PLGA biblock polymer) is about 37% at the tenth day. As shown in FIG. 2B, at the 17^{th}-20^{th} days, the slope of the cumulative release amount curve of TGF-b1 in 100% PRP sample solution (without the mPEG-PLGA biblock polymer) is substantially zero. Furthermore, the release amount of TGF-b1 in 100% PRP sample solution (without the mPEG-PLGA biblock polymer) is increased about 2%. It proves that the PRP covered by mPEG-PLGA biblock polymer exhibits slow release effect.

### Example 2

Test of Transforming Growth Factor (TGF-b1) Activity Against Human Dermal Fibroblasts Cell (HDF) Proliferation Rate

0.2 mL of culture fluid was extracted from the cell culture plates of Preparation Examples 8-11 individually after reacting 24 hr. Next, 0.2 mL of medium (DMEM) was implanted in the cell culture plate of Preparation Examples 8-11. After reacting another day, the agent (alamar blue assay, sold by Bio-Rad) was added into the cell culture plate. After reacting, the human dermal fibroblasts cell (HDF) proliferation rate of the result was determined by measuring absorbance at 570 nm by a spectrophotometer.

### Example 3

0.2 mL of culture fluid was extracted from the cell culture plates of Preparation Examples 8-11 individually after reacting 24 hr. Next, 0.2 mL of medium (DMEM) was implanted in the cell culture plate of Preparation Examples 8-11. The aforementioned steps were repeated until 5 days. The agent (alamar blue assay, sold by Bio-Rad) was added into the cell culture plate. After reacting, the human dermal fibroblasts cell (HDF) proliferation rate of the result was determined by measuring absorbance at 570 nm by a spectrophotometer.

### Example 4

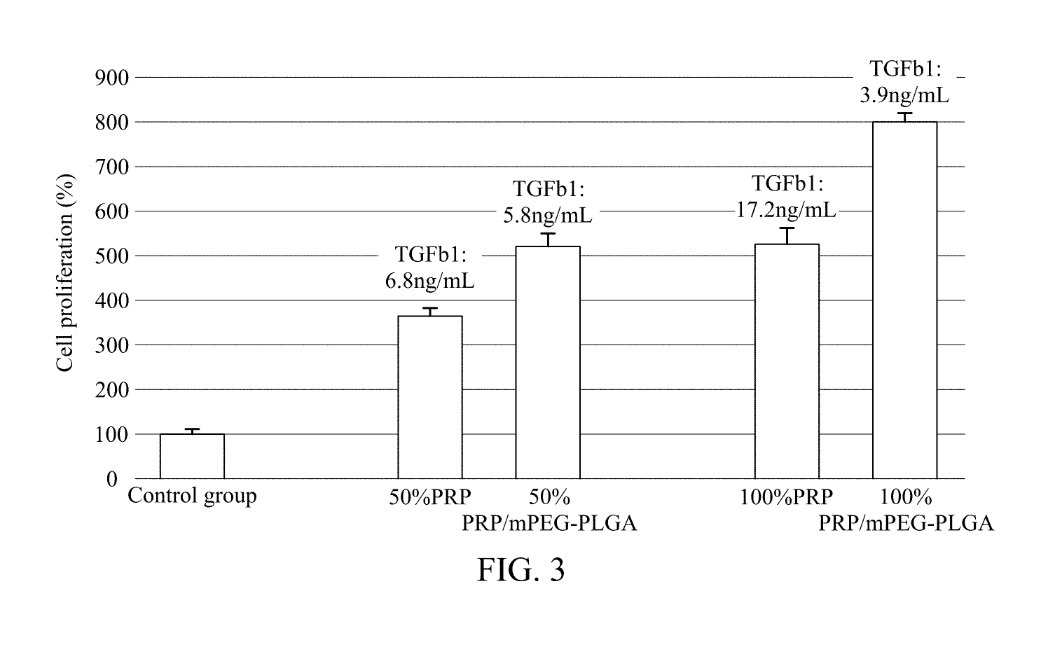

0.2 mL of culture fluid was extracted from the cell culture plates of Preparation Examples 8-11 individually after reacting 24 hr. Next, 0.2 mL of medium (DMEM) was implanted in the cell culture plate of Preparation Examples 8-11. The aforementioned steps were repeated until 5 days. The agent (alamar blue assay, sold by Bio-Rad) was added into the cell culture plate. After reacting, the human dermal fibroblasts cell (HDF) proliferation rate of the result was determined by measuring absorbance at 570 nm by a spectrophotometer. In the meantime, the clear supernatant liquid of culture fluid was extracted, and the concentration of TGF-b1 of the clear supernatant liquid was determined by enzyme-linked immunosorbent assay (ELISA), and the result is shown in FIG. 3.

As shown in FIG. 3, whether the concentration of PRP sample solution (including the mPEG-PLGA biblock polymer) is 50% or 100%, the release amount or activity of the transforming growth factor would not be affected by the mPEG-PLGA biblock polymer. The release amount of TGF-b1 in 50% PRP sample solution (without the mPEG-PLGA biblock polymer) was about 6.8 ng/ml, and the human dermal fibroblasts cell (HDF) proliferation rate of 50% PRP sample solution (without the mPEG-PLGA biblock polymer) was about 370%. In the presence of mPEG-PLGA biblock polymer, although the release amount of TGF-b1 in 50% PRP sample solution (including the mPEG-PLGA biblock polymer) was reduced to about 5.8 ng/ml, and the human dermal fibroblasts cell (HDF) proliferation rate of 50% PRP sample solution (without the mPEG-PLGA biblock polymer) was increased to about 531%. In 100% PRP sample solution, a similar result was observed. The release amount of TGF-b1 in 50% PRP sample solution (without the mPEG-PLGA biblock polymer) was about 17.2 ng/ml, and the human dermal fibroblasts cell (HDF) proliferation rate of 50% PRP sample solution (without the mPEG-PLGA biblock polymer) was about 537%. In the presence of mPEG-PLGA biblock polymer, although the release amount of TGF-b1 in 50% PRP sample solution (including the mPEG-PLGA biblock polymer) was reduced to about 3.9 ng/ml, and the human dermal fibroblasts cell (HDF) proliferation rate of 50% PRP sample solution (without the mPEG-PLGA biblock polymer) was sharply increased to about 797%. Accordingly, in the absence of the slow release effect of mPEG-PLGA biblock polymer. TGF-b1 would be released largely, thereby inhibiting the proliferation of human dermal fibroblasts cell (HDF). Transforming growth factor TGF-b1, with suitable amount, facilitates the proliferation of human dermal fibroblasts cell (HDF).

### Example 5

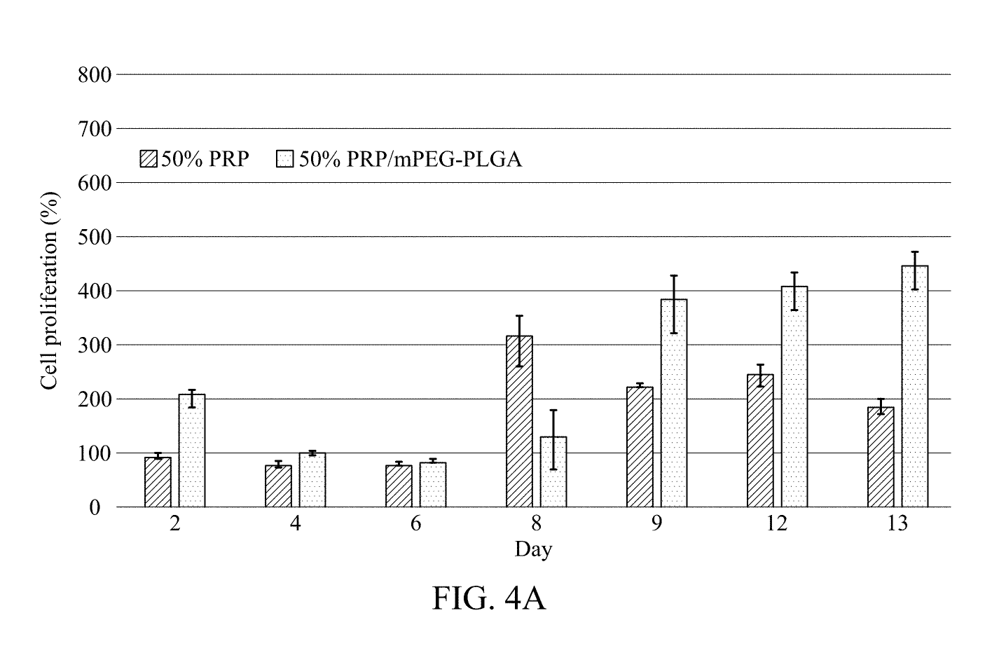

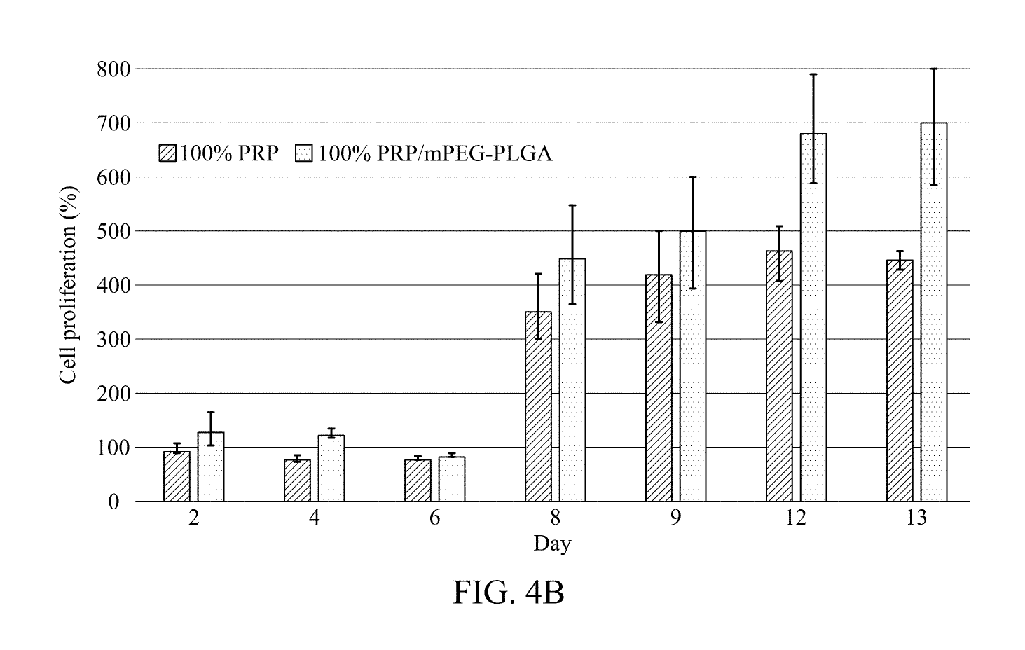

0.6 mL of culture fluid was extracted from the cell culture plates of Preparation Examples 12-15 individually after reacting 24 hr, and then stored at −20° C. Next, 0.6 mL of medium (DMEM) was implanted in the cell culture plate of Preparation Examples 12-15, and then 0.6 mL of culture fluid was extracted from the cell culture plates of Preparation Examples 12-15 individually after reacting 24 hr, and then stored at −20° C. The aforementioned steps were repeated until 13 days (analyzed at the second, fourth, sixth, eighth, ninth, twelfth and thirteenth days). After implanting human dermal fibroblasts cell (HDF) into a 96 well plate, the above sample solutions (0.1 mL) were added into the 96 well plate individually. After standing at a cell incubator for 72 hr, the agent (alamar blue assay) was added into the 96 well plate. The human dermal fibroblasts cell (HDF) proliferation rate of the results were determined by measuring absorbance at 570 nm by a spectrophotometer, and the results are shown in FIGS. 4A and 4B.

As shown in FIGS. 4A and 4B, whether the concentration of PRP sample solution (including the mPEG-PLGA biblock polymer) is 50% or 100%, the release amount or activity of the transforming growth factor would not be affected by the mPEG-PLGA biblock polymer. Furthermore, the released transforming growth factor (TGF-b1) facilitates the proliferation of human dermal fibroblasts cell (HDF).

### Test of PRP Activity Preservation

### Example 6

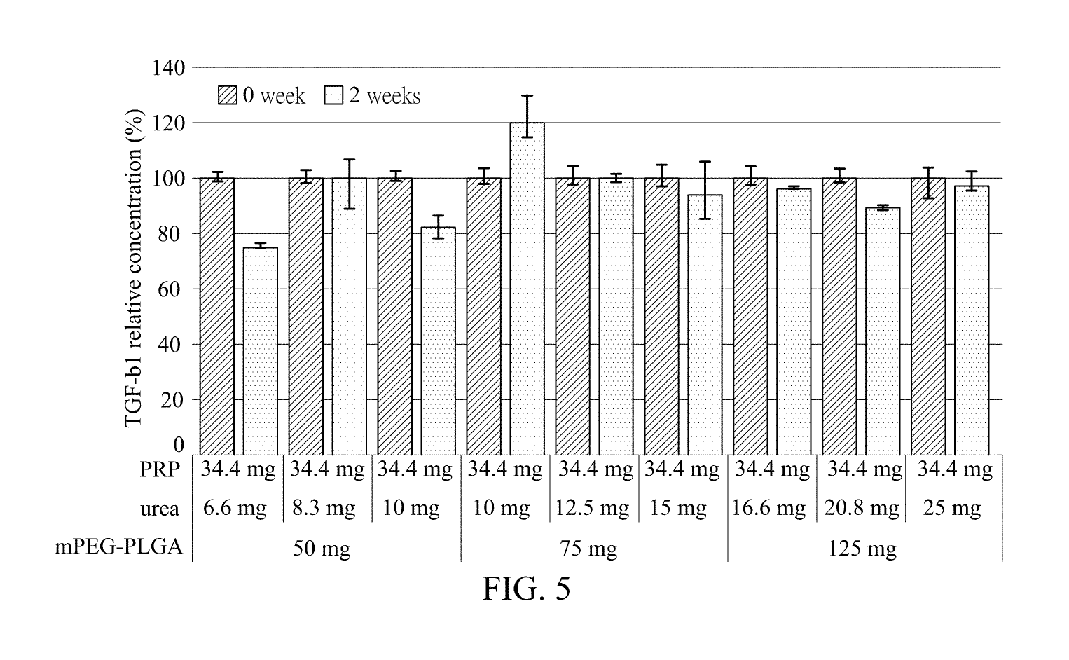

50 mg of mPEG-PLGA-BOX-PLGA-mPEG polymer, 6.6 mg of urea, and 34.4 mg of 100% PRP sample solution (without the mPEG-PLGA biblock polymer) were dissolved in water and stirred at 4° C., obtaining a solution. Next, the aforementioned solution was cooled to −20° C. to form a solid mixture. Finally, the solid mixture was subjected to a freeze-drying process at −20° C. under 8 mTorr for 2 days, obtaining a powder form. The powder form was stored at room temperature and −20° C. individually. After two weeks, the powder forms (stored at room temperature and stored at −20° C.) were dissolved in 409 mg of water individually, and then the release amount of the TGF-b1 of the results was determined by enzyme-linked immunosorbent assay (ELISA), and the results are shown in FIG. 5.

### Example 7

50 mg of mPEG-PLGA-BOX-PLGA-mPEG polymer, 8.3 mg of urea, and 34.4 mg of 100% PRP sample solution (without the mPEG-PLGA biblock polymer) were dissolved in water and stirred at 4° C., obtaining a solution. Next, the aforementioned solution was cooled to −20° C. to form a solid mixture. Finally, the solid mixture was subjected to a freeze-drying process at −20° C. under 8 mTorr for 2 days, obtaining a powder form. The powder form was stored at room temperature and −20° C. individually. After two weeks, the powder forms (stored at room temperature and stored at −20° C.) were dissolved in 407.3 mg of water individually, and then the release amount of the TGF-b1 of the results was determined by enzyme-linked immunosorbent assay (ELISA), and the results are shown in FIG. 5.

### Example 8

50 mg of mPEG-PLGA-BOX-PLGA-mPEG polymer, 10 mg of urea, and 34.4 mg of 100% PRP sample solution (without the mPEG-PLGA biblock polymer) were dissolved in water and stirred at 4° C., obtaining a solution. Next, the aforementioned solution was cooled to −20° C. to form a solid mixture. Finally, the solid mixture was subjected to a freeze-drying process at −20° C. under 8 mTorr for 2 days, obtaining a powder form. The powder form was stored at room temperature and −20° C. individually. After two weeks, the powder forms (stored at room temperature and stored at −20° C.) were dissolved in 405.6 mg of water individually, and then the release amount of the TGF-b1 of the results was determined by enzyme-linked immunosorbent assay (ELISA), and the results are shown in FIG. 5.

### Example 9

75 mg of mPEG-PLGA-BOX-PLGA-mPEG polymer, 10 mg of urea, and 34.4 mg of 100% PRP sample solution (without the mPEG-PLGA biblock polymer) were dissolved in water and stirred at 4° C., obtaining a solution. Next, the aforementioned solution was cooled to −20° C. to form a solid mixture. Finally, the solid mixture was subjected to a freeze-drying process at −20° C. under 8 mTorr for 2 days, obtaining a powder form. The powder form was stored at room temperature and −20° C. individually. After two weeks, the powder forms (stored at room temperature and stored at −20° C.) were dissolved in 380.6 mg of water individually, and then the release amount of the TGF-b1 of the results was determined by enzyme-linked immunosorbent assay (ELISA), and the results are shown in FIG. 5.

### Example 10

75 mg of mPEG-PLGA-BOX-PLGA-mPEG polymer, 12.5 mg of urea, and 34.4 mg of 100% PRP sample solution (without the mPEG-PLGA biblock polymer) were dissolved in water and stirred at 4° C., obtaining a solution. Next, the aforementioned solution was cooled to −20° C. to form a solid mixture. Finally, the solid mixture was subjected to a freeze-drying process at −20° C. under 8 mTorr for 2 days, obtaining a powder form. The powder form was stored at room temperature and −20° C. individually. After two weeks, the powder forms (stored at room temperature and stored at −20° C.) were dissolved in 378.1 mg of water individually, and then the release amount of the TGF-b1 of the results was determined by enzyme-linked immunosorbent assay (ELISA), and the results are shown in FIG. 5.

### Example 11

75 mg of mPEG-PLGA-BOX-PLGA-mPEG polymer, 15 mg of urea, and 34.4 mg of 100% PRP sample solution (without the mPEG-PLGA biblock polymer) were dissolved in water and stirred at 4° C., obtaining a solution. Next, the aforementioned solution was cooled to −20° C. to form a solid mixture. Finally, the solid mixture was subjected to a freeze-drying process at −20° C. under 8 mTorr for 2 days, obtaining a powder form. The powder form was stored at room temperature and −20° C. individually. After two weeks, the powder forms (stored at room temperature and stored at −20° C.) were dissolved in 375.6 mg of water individually, and then the release amount of the TGF-b1 of the results was determined by enzyme-linked immunosorbent assay (ELISA), and the results are shown in FIG. 5.

### Example 12

125 mg of mPEG-PLGA-BOX-PLGA-mPEG polymer, 16.6 mg of urea, and 34.4 mg of 100% PRP sample solution (without the mPEG-PLGA biblock polymer) were dissolved in water and stirred at 4° C., obtaining a solution. Next, the aforementioned solution was cooled to −20° C. to form a solid mixture. Finally, the solid mixture was subjected to a freeze-drying process at −20° C. under 8 mTorr for 2 days, obtaining a powder form. The powder form was stored at room temperature and −20° C. individually. After two weeks, the powder forms (stored at room temperature and stored at −20° C.) were dissolved in 324 mg of water individually, and then the release amount of the TGF-b1 of the results was determined by enzyme-linked immunosorbent assay (ELISA), and the results are shown in FIG. 5.

### Example 13

125 mg of mPEG-PLGA-BOX-PLGA-mPEG polymer, 20.8 mg of urea, and 34.4 mg of 100% PRP sample solution (without the mPEG-PLGA biblock polymer) were dissolved in water and stirred at 4° C., obtaining a solution. Next, the aforementioned solution was cooled to −20° C. to form a solid mixture. Finally, the solid mixture was subjected to a freeze-drying process at −20° C. under 8 mTorr for 2 days, obtaining a powder form. The powder form was stored at room temperature and −20° C. individually. After two weeks, the powder forms (stored at room temperature and stored at −20° C.) were dissolved in 319.8 mg of water individually, and then the release amount of the TGF-b1 of the results was determined by enzyme-linked immunosorbent assay (ELISA), and the results are shown in FIG. 5.

### Example 14

125 mg of mPEG-PLGA-BOX-PLGA-mPEG polymer, 25 mg of urea, and 34.4 mg of 100% PRP sample solution (without the mPEG-PLGA biblock polymer) were dissolved in water and stirred at 4° C., obtaining a solution. Next, the aforementioned solution was cooled to −20° C. to form a solid mixture. Finally, the solid mixture was subjected to a freeze-drying process at −20° C. under 8 mTorr for 2 days, obtaining a powder form. The powder form was stored at room temperature and −20° C. individually. After two weeks, the powder forms (stored at room temperature and stored at −20° C.) were dissolved in 315.6 mg of water individually, and then the release amount of the TGF-b1 of the results was determined by enzyme-linked immunosorbent assay (ELISA), and the results are shown in FIG. 5.

As shown in FIG. 5, PRP (mixed with mPEG-PLGA biblock polymer and urea under a specific ratio) solution, which was stored at room temperature after two weeks, had a TGF-b1 concentration similar to that of PRP solution, which was just prepared. It proves that mPEG-PLGA biblock polymer could preserve the activity of PRP at room temperature.

### Example 15

Activity Released Test of Transforming Growth Factor (TGF-b1) Preserved at Room Temperature

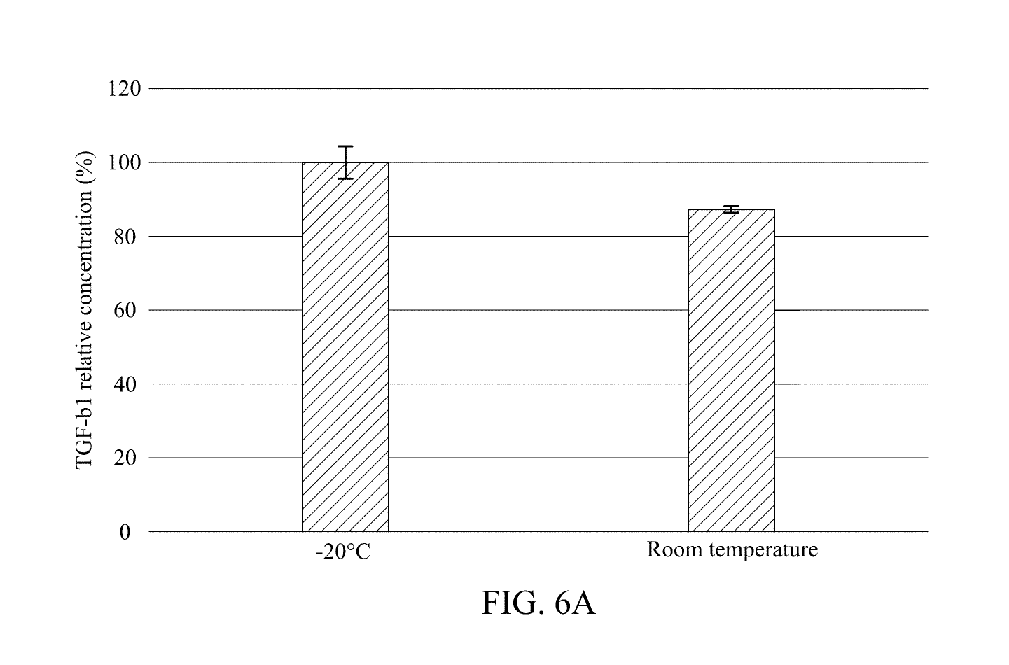

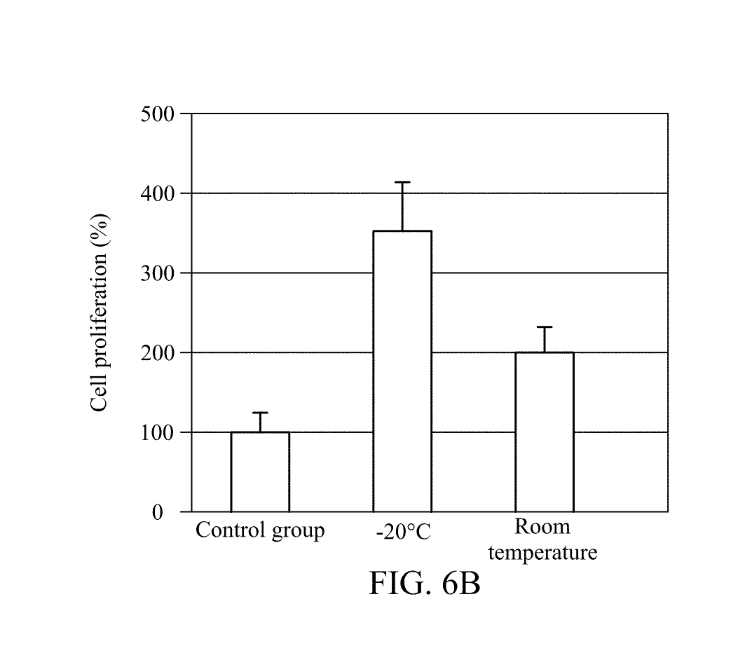

Platelet-rich plasma (PRP) of Preparation Example 1 was cooled to −20° C. to form a powder form. The powder form was stored at room temperature and −20° C. individually. After one month, the powder forms (stored at room temperature and stored at −20° C.) were dissolved in water individually, and then the release amount of the TGF-b1 of the results was determined by enzyme-linked immunosorbent assay (ELISA), and the results are shown in FIG. 6A. The result shows that the TGF-b1 concentration of the powder form stored at room temperature is 10% less than that of the powder form stored at −20° C.

### Example 16

Test of Transforming Growth Factor (TGF-b1) Activity Against Human Dermal Fibroblasts Cell (HDF) Proliferation Rate at Room Temperature

Platelet-rich plasma (PRP) of Preparation Example 1 was cooled to −20° C. to form a powder form. The powder form was stored at room temperature and −20° C. individually. After one month, the powder forms (stored at room temperature and stored at −20° C.) were dissolved in water individually. After mixing with the agent (alamar blue assay) and then reacting, the human dermal fibroblasts cell (HDF) proliferation rate of the result was determined by measuring absorbance at 570 nm by a spectrophotometer, and the result is shown in FIG. 6b. The result shows that the TGF-b1 of the powder form stored at room temperature could be released. In comparison with the control group (i.e. without PRP), the solution prepared from the powder form could facilitate the proliferation of human dermal fibroblasts cell (HDF).

### Example 17

Slow Release Test of Platelet-Rich Plasma (PRP) with Various mPEG-PLGA Biblock Polymer Concentration

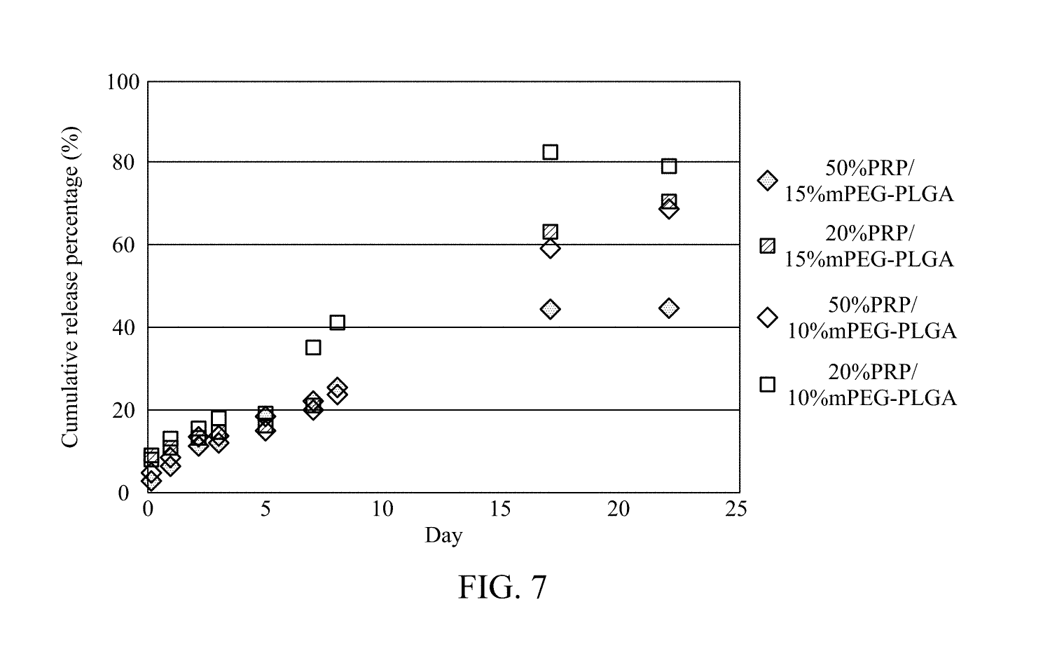

mPEG-PLGA biblock polymer (dissolved in water) was mixed with platelet-rich plasma (PRP) to prepare PRP solution with various concentration. mPEG-PLGA and platelet-rich plasma were mixed uniformly at 25° C. Next, the mixture was disposed into a specific release element, and 500 μL of the mixture was released by the release element at predetermined time instants for measuring the cumulative release amount and release rate of TGF-b1. The result is shown in FIG. 7. As shown in FIG. 7, the composition including 10% mPEG-PLGA and PRP could form a gel. As shown in FIG. 7, the result of 22 days test was similar to the result of the 7 day-test. Namely, the release rate of 10% mPEG-PLGA was better than that of 15% mPEG-PLG. Furthermore, the release rate of 20% PRP was better than that of 50% PRP. On the 17^{th} day, each group released TGF-b continuously (as indicated by the arrow). On the 22^{nd} day, except for the group of 20% PRP/10% mPEG-PLGA, each group did not obviously release TGF-b1.

### Example 18

In Vitro Releasing Test of mPEG-PLGA Biblock Polymer

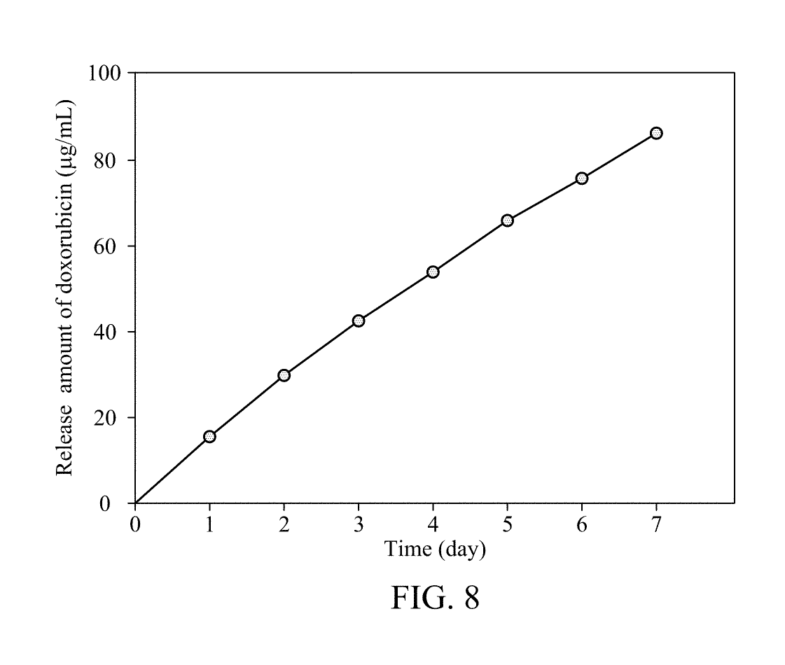

First, 1 mg/mL of doxorubicin was added into 15% mPEG-PLGA aqueous solution, and the mixture was stirred at 25° C. for uniformly mixing mPEG-PLGA and doxorubicin. Next, 1 mL of the result was disposed on the bottom of the release element (10 mL), and then heated at 37° C. for 5 min to form a gel. Next, 9 mL of phosphate buffered saline (PBS) was added into the release element, and the release element was disposed on a thermostat (37° C.) and shaken at 50 rpm, obtaining a PBS-miscible solution. A small amount of PBS-miscible solution was analyzed by HPLC to determine the release ratio of doxorubicin each day, and the result is shown in FIG. 8. As shown in FIG. 8, after mixing doxorubicin (small molecule drug) and 15% mPEG-PLGA, the release amount of the mixture was about 20 g/ml after one day. This means that mPEG-PLGA could exhibit a good coating effect.

### Example 19

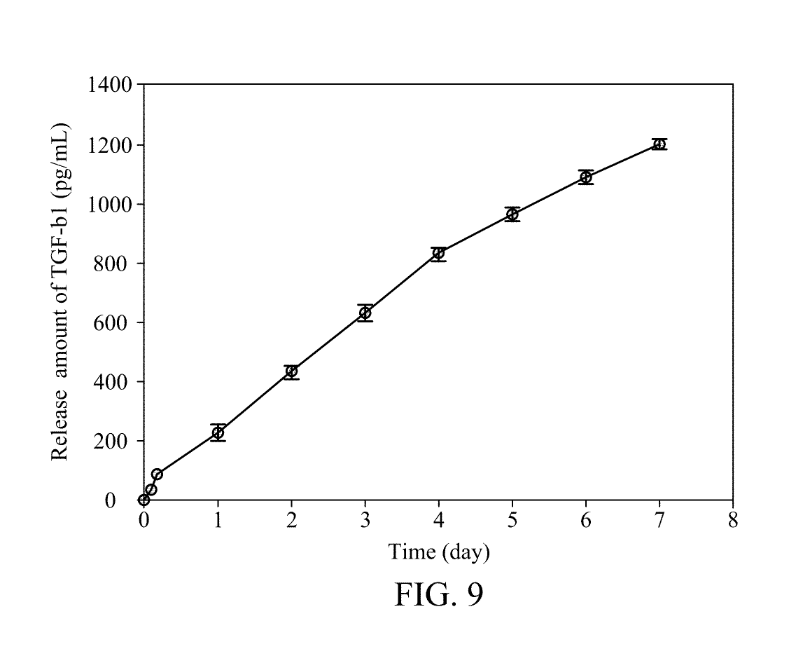

First, TGF-b1 was added into 15% mPEG-PLGA aqueous solution, and the mixture was stirred at 25° C. for uniformly mixing mPEG-PLGA and TGF-b1. Next, 1 mL of the result was disposed on the bottom of the release element (10 mL), and then heated at 37° C. for 5 min to form a gel. Next, 9 mL of phosphate buffered saline (PBS) was added into the release element, and the release element was disposed on a thermostat (37° C.) and shaken at 50 rpm, obtaining a PBS-miscible solution. A small amount of PBS-miscible solution was analyzed by HPLC to determine the release ratio of TGF-b1 each day, and the result is shown in FIG. 9. As shown in FIG. 9, after mixing TGF-b1 and 15% mPEG-PLGA for one day, TGF-b1 would not be released largely. This means that mPEG-PLGA could exhibit a good coating effect. After seven days, the results show that TGF-b1 covered by mPEG-PLGA biblock polymer exhibited a slow release effect.

### Example 20

Releasing Test of Ultrasound Stimulation

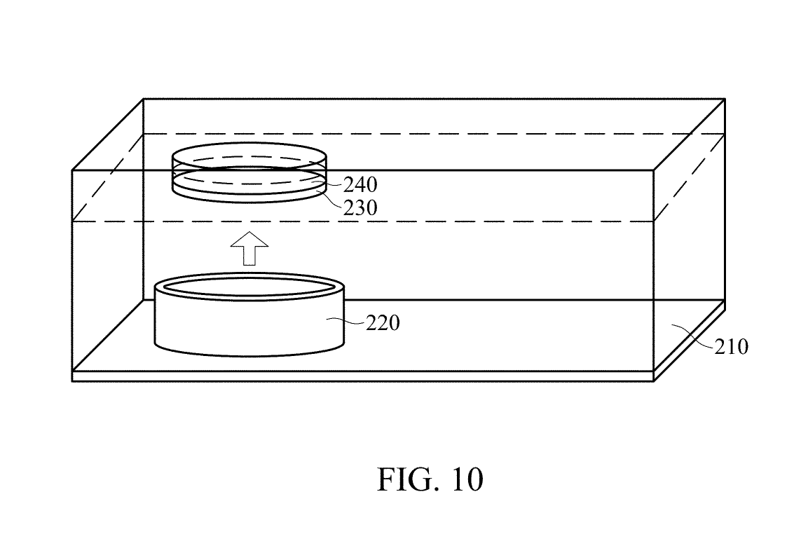

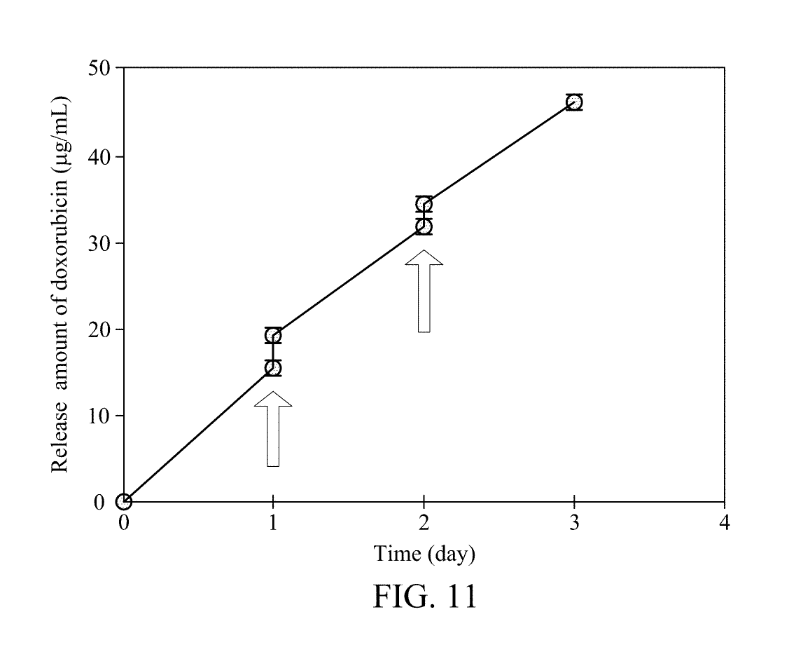

First, doxorubicin was added into a 15% mPEG-PLGA aqueous solution (the concentration of doxorubicin was 1 mg/mL). The mixture was stirred at 25° C. for uniformly mixing mPEG-PLGA 230 and doxorubicin. Next, 1 mL of the result was disposed on the bottom of the release element 240 (10 mL), and then heated at 37° C. for 5 min to form a gel 230. Next. 9 mL of phosphate buffered saline (PBS) was added into the release element, and the release element was disposed on a thermostat 210 (37° C.) and shaken at 50 rpm, obtaining a PBS-miscible solution. At predetermined time instants. PBS-miscible solution was subjected to an ultrasound stimulation (as indicated by the arrow) via an ultrasound stimulation element 220 (as shown in FIG. 10) for 5 min. 500 μL of the PBS-miscible solution was released by the release element 240 and then the doxorubicin concentration of PBS-miscible solution was determined by enzyme-linked immunosorbent assay (ELISA).

### Example 21

The PBS-miscible solution of Example 18 was provided. The conditions with ultrasound stimulation or without ultrasound stimulation were compared. As shown in FIG. 1I, the arrow represents the ultrasound stimulation. On the premise of the same release time period, the PBS-miscible solution (without ultrasound stimulation) had a doxorubicin concentration of about 15 μg/mL, and the PBS-miscible solution (with ultrasound stimulation) had a doxorubicin concentration of about 45 μg/mL. As a result, the doxorubicin release rate of the PBS-miscible solution with ultrasound stimulation was three times higher than that of the PBS-miscible solution without ultrasound stimulation. This means that the release amount of doxorubicin could be controlled when covering doxorubicin with mPEG-PLGA, as shown in FIG. 1I.

### Example 22

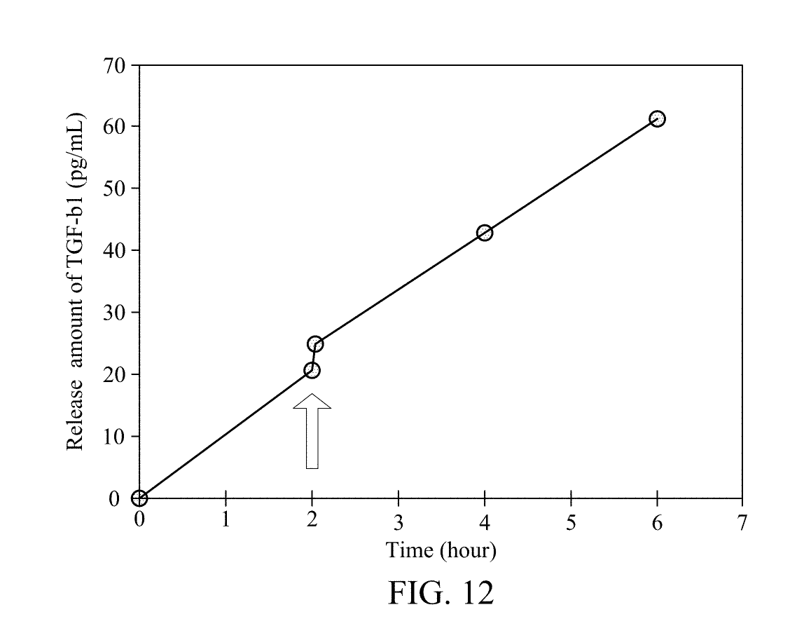

The PBS-miscible solution of Example 19 was provided. At predetermined time instants, PBS-miscible solution was subjected to an ultrasound stimulation (as indicated by the arrow) via an ultrasound stimulation element 220 (as shown in FIG. 10) for 5 min. Next. 500 μL of the PBS-miscible solution was released by the release element 240 and then the TGF-b1 concentration of PBS-miscible solution was determined by enzyme-linked immunosorbent assay (ELISA), and the result is shown in FIG. 12. FIG. 12 shows the release amount of TGF-b1 covered by mPEG-PLGA. The arrow represents the ultrasound stimulation. On the premise of the same release time period, the PBS-miscible solution (without ultrasound stimulation) had a TGF-b1 concentration of about 20 pg/mL, and the PBS-miscible solution (with ultrasound stimulation) had a TGF-b1 concentration of about 60 pg/mL. As a result, the TGF-b1 release rate of the PBS-miscible solution with ultrasound stimulation was 3 times higher than that of the PBS-miscible solution without ultrasound stimulation. This means that the release amount of TGF-b1 could be controlled when covering doxorubicin with mPEG-PLGA.

### Example 23

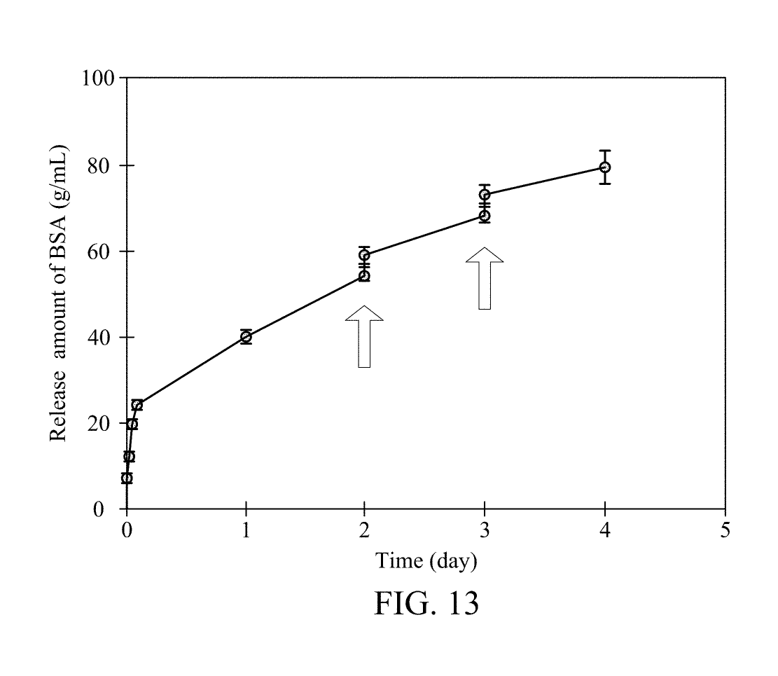

First, bovine serum albumin (BSA) was added into 15% mPEG-PLGA aqueous solution. The mixture was stirred at 25° C. for uniformly mixing mPEG-PLGA 230 and bovine serum albumin. Next, 1 mL of the result was disposed on the bottom of the release element (10 mL), and then heated at 37° C. for 5 min to form a gel. Next, 9 mL of phosphate buffered saline (PBS) was added into the release element, and the release element was disposed on a thermostat 210 (37° C.) and shaken at 50 rpm, obtaining a PBS-miscible solution. At predetermined time instants, PBS-miscible solution was subjected to an ultrasound stimulation (as indicated by the arrow) via an ultrasound stimulation element 220 (as shown in FIG. 10) for 5 min. 500 μL of the PBS-miscible solution was released by the release element 240 and then the bovine serum albumin concentration of PBS-miscible solution was determined by enzyme-linked immunosorbent assay (ELISA), and the result is shown in FIG. 13. As shown in FIG. 13, on the premise of the same release time period, the PBS-miscible solution (without ultrasound stimulation) had a bovine serum albumin concentration of about 55 g/mL, and the PBS-miscible solution (with ultrasound stimulation (as indicated by the arrow)) had a bovine serum albumin concentration of about 80 g/mL. As a result, the bovine serum albumin release rate of the PBS-miscible solution with ultrasound stimulation was 1.45 times higher than that of the PBS-miscible solution without ultrasound stimulation. This means that the release amount of a large molecule drug (such as bovine serum albumin) could be controlled when covering the large molecule drug with mPEG-PLGA.

### Comparative Example 1

Stagnation Test of Drug-Containing Hydrogel Against Hyaluronic Acid

10% mPEG-PLGA was mixed with doxorubicin to form a drug-containing hydrogel (the doxorubicin concentration was 1 mg/mL). 2 mL of hyaluronic acid (sold by Hyalgan) and the drug-containing hydrogel were subjected to stagnation tests. The stagnation test included following steps. The hyaluronic acid and the drug-containing hydrogel were injected onto an inclined plate with a slop of 15 degree, and the flowing situations of the hyaluronic acid and the drug-containing hydrogel were observed. As a result, the hyaluronic acid was flowable at 37° C., and the drug-containing hydrogel was gel and not flowable. Therefore, the drug-containing hydrogel could be trapped in the affected area, thereby extending the drug effect.

### Comparative Example 2

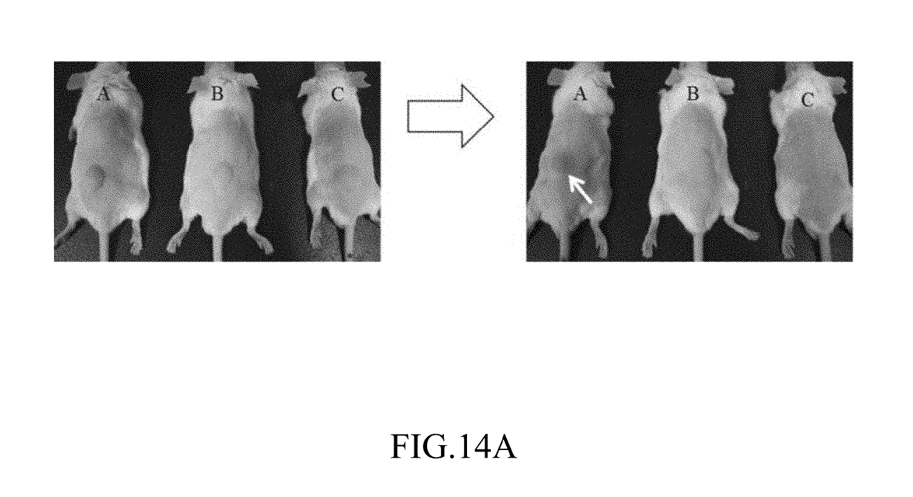

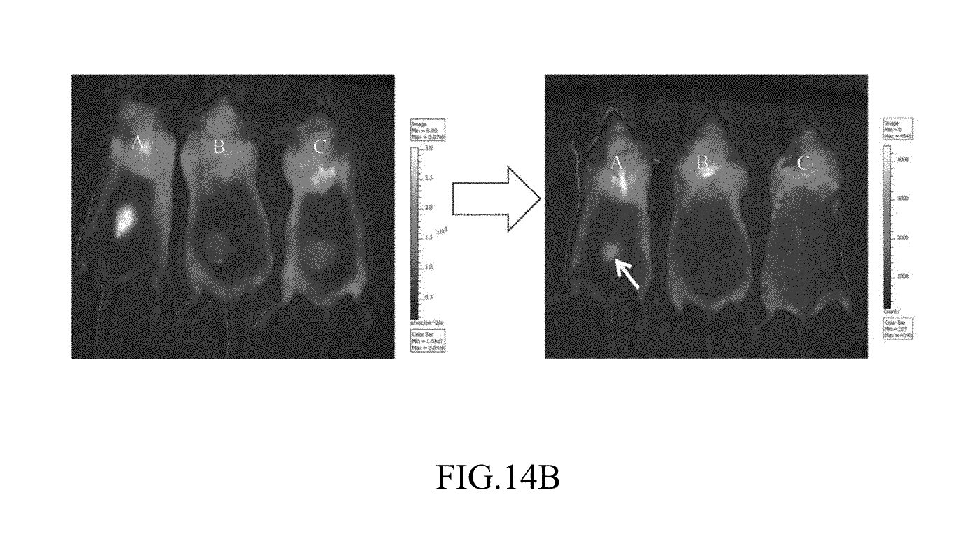

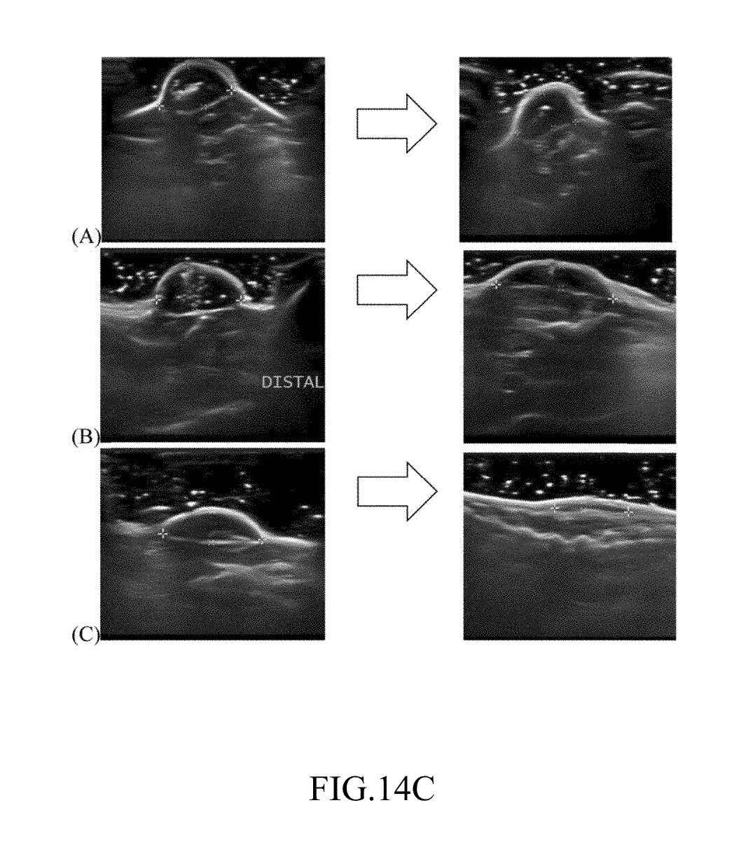

150 μL of the drug-containing hydrogel (A) (10% mPEG-PLGA/Doxorubicin, wherein the doxorubicin concentration was 1 mg/mL), the hyaluronic acid (B) (sold by Hyalgan), and the saline solution (C) were injected into the subcutaneous tissue of mice individually. The visual appearances of mice was observed via visual inspection, and the stagnation condition in the subcutaneous tissue was imaged by ultrasonic detection. Finally, the fluorescence intensity was detected by IVIS spectrum. After seven days, the subcutaneous mass (as indicated by the arrow), which the drug-containing hydrogel (A) was injected thereinto, had a relatively high drug retention, as shown in FIG. 14. The blood test confirmed the effect of sustained release to systemic circulation.

The drug-containing hydrogel (A) (10% mPEG-PLGA/Doxorubicin, wherein the doxorubicin concentration was 1 mg/mL), the hyaluronic acid (B) (sold by Hyalgan), and the saline solution (C) were injected into the subcutaneous tissue of mice individually. FIG. 14B shows the flowing situations and performance in vivo via IVIS spectrum. As shown in FIG. 14B, 10% mPEG-PLGA/Doxorubicin was still observed (as indicated by the arrow) after seven days. FIG. 14C shows the result which was observed by ultrasonic imaging. The drug-containing hydrogel (A) (10% mPEG-PLGA/Doxorubicin, wherein the doxorubicin concentration was 1 mg/mL), the hyaluronic acid (B) (sold by Hyalgan), and the saline solution (C) were injected into the subcutaneous tissue of mice individually. As shown in FIG. 14C, the subcutaneous tissue (detected by ultrasonic imaging), which the drug-containing hydrogel (A) was injected thereinto, had a relatively high drug retention (elliptical bump). The subcutaneous tissue, which the hyaluronic acid (B) was injected thereinto, was relatively flat. The subcutaneous tissue, which the saline solution (c) was injected thereinto, was almost nonexistent in the original location.

The mPEG-PLGA biblock polymer of the disclosure has high thermal-sensitivity. The convenience of using the mPEG-PLGA biblock polymer can be improved by modifying the components and amounts. Drugs can be fixed in specific regions by means of the mPEG-PLGA biblock polymer to avoid drug flow. The mPEG-PLGA biblock polymer can be turned into a gel by body temperature by means of the advantage of phase change in the human body (37° C.) gel. Furthermore, drugs or transforming growth factor can be affixed to the wound tissue by means of the mPEG-PLGA biblock polymer, thereby effectively promoting tissue healing and restoring tissue integrity.

In addition, according to the Examples of the disclosure, the mPEG-PLGA biblock polymer can effectively cover doxorubicin, bovine serum albumin, or transforming growth factor. Furthermore, the release amount of the aforementioned substances can be increased 1.45-3 times via ultrasound stimulation. This means that the mPEG-PLGA biblock polymer exhibits a controllable release ability.

It will be clear that various modifications and variations can be made to the disclosed methods and materials. It is intended that the specification and examples be considered as exemplary only, with the true scope of the disclosure being indicated by the following claims and their equivalents.

What is claimed is:

1. A powder form, wherein the powder form consists essentially of 120-380 parts by weight of biodegradable copolymer, 15-75 parts by weight of urea, and 100 parts by weight of platelet-rich plasma (PRP), wherein the biodegradable copolymer has a structure of Formula (I) or Formula (II): 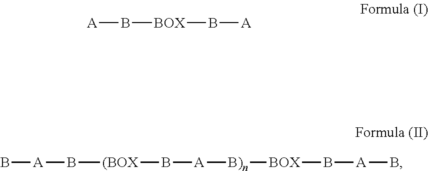
wherein A is a hydrophilic polyethylene glycol polymer;
B is a hydrophobic polyester polymer;
BOX is a bifunctional group monomer of 2, 2′-bis(2-oxazoline) used for coupling the blocks A-B or B-A-B; and
n is 0 or an integer greater than 0.

2. The powder form as claimed in claim 1, wherein A comprises polyethylene glycol (PEG), or methoxy-poly(ethylene glycol (mPEG).

3. The powder form as claimed in claim 1, wherein the B comprises poly(lactide-co-glycolide) (PLGA), poly(propionic-co-lactic (PPLA), poly(valeric-co-lactic) (PVLA), or poly(caproic-co-lactic (PCLA).

4. A use of hydrogel composition for alleviating degenerative joint and tendon tear, wherein the hydrogel composition comprises 100 parts by weight of therapeutic agent and 120-380 parts by weight of biodegradable copolymer, wherein the therapeutic agent comprises platelet-rich plasma (PRP), doxorubicin, transforming growth factor, bovine serum albumin, or a combination thereof, wherein the biodegradable copolymer has a structure of Formula (I) or Formula (II): 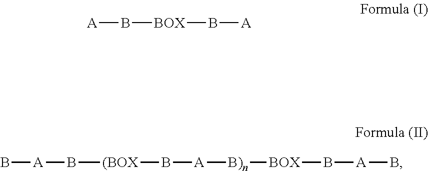
wherein A is a hydrophilic polyethylene glycol polymer:
B is a hydrophobic polyester polymer;
BOX is a bifunctional group monomer of 2, 2′-bis(2-oxazoline) used for coupling the blocks A-B or B-A-B; and
n is 0 or an integer greater than 0.

5. The use of hydrogel composition for alleviating degenerative joint and tendon tear as claimed in claim 4, wherein A comprises polyethylene glycol (PEG), or methoxy-poly(ethylene glycol (mPEG).

6. The use of hydrogel composition for alleviating degenerative joint and tendon tear as claimed in claim 4, wherein the B comprises poly(lactide-co-glycolide) (PLGA), poly(propionic-co-lactic (PPLA), poly(valeric-co-lactic) (PVLA), or poly(caproic-co-lactic (PCLA).

7. The use of hydrogel composition for alleviating degenerative joint and tendon tear as claimed in claim 4, wherein the therapeutic agent comprises platelet-rich plasma (PRP) and doxorubicin, wherein the weight ratio of the platelet-rich plasma (PRP) to the doxorubicin is 1:2.

8. The use of hydrogel composition for alleviating degenerative joint and tendon tear as claimed in claim 4, wherein the therapeutic agent comprises platelet-rich plasma (PRP) and transforming growth factor, wherein the weight ratio of the platelet-rich plasma (PRP) to the transforming growth factor is 1:2.

9. The use of hydrogel composition for alleviating degenerative joint and tendon tear as claimed in claim 4, wherein the therapeutic agent comprises platelet-rich plasma (PRP) AND bovine serum albumin, wherein the weight ratio of the platelet-rich plasma (PRP) to the bovine serum albumin is 1:2.

10. The use of hydrogel composition for alleviating degenerative joint and tendon tear as claimed in claim 4, wherein the use of hydrogel composition further comprises an ultrasonic treatment, which forces the hydrogel composition to release the therapeutic agent.

11. The use of hydrogel composition for alleviating degenerative joint and tendon tear as claimed in claim 10, wherein the output intensity of the ultrasonic treatment is 100-10000 W/cm².

12. The use of hydrogel composition for alleviating degenerative joint and tendon tear as claimed in claim 10, wherein the ultrasonic treatment has a treatment period from 1 min to 5 min.

13. The use of hydrogel composition for alleviating degenerative joint and tendon tear as claimed in claim 4, wherein the hydrogel composition further comprises 50-400 parts by weight of water.
